(19) United States
(12) Patent Application Publication
(10) Pub. No.: US 2024/0029828 A1
(43) Pub. Date: Jan. 25, 2024

(54) Computational Method and System for Compression of Genetic Information

(71) Applicant: THE REGENTS OF THE UNIVERSITY OF CALIFORNIA, Oakland, CA (US)

(72) Inventors: David Frank Savage, Berkeley, CA (US); John James Desmarais, Berkeley, CA (US); Luke Mcdonald Oltrogge, Berkeley, CA (US); Abraham Isser Flamholz, Berkeley, CA (US)

(21) Appl. No.: 18/266,111

(22) PCT Filed: Dec. 9, 2021
(86) PCT No.: PCT/US21/62573

§ 371 Date: Jun. 8, 2023

Related U.S. Application Data
(60) Provisional application No. 63/123,884, filed on Dec. 10, 2020.

(51) Int. Cl. G16B 30/00; G16B 50/50

(52) U.S. Cl. CPC G16B 30/00; G16B 50/50

(57) ABSTRACT

To reduce the total amount of linear sequence (DNA, RNA or other medium) required to encode a set of genetic elements, the present disclosure describes a computational method for compressing genetic information by finding one or more sequences that each mutually encode multiple genetic elements in the same stretch of sequence (a “co-encoding”). The computational method encodes each of the genetic elements in respective directed acyclic graphs (DAGs) or finite automatons (FAs), then encodes overlapping sequences between the DAGs or FAs in a second DAG or FA. Additional DAGs or FAs may be encoded for overlapping sequences that result from shifting the reading frame of the genetic elements relative to one another and switching the orientation of the elements.

### CROSS REFERENCE TO RELATED APPLICATION

This application is a national phase application under 35 U.S.C. § 371 of international application PCT/US2021/062573, filed Dec. 9, 2021, which claims the benefit of priority under 35 U.S.C. § 119 to U.S. Provisional Patent Application No. 63/123,884, filed on Dec. 10, 2020, the disclosure of which is hereby incorporated by reference in its entirety.

### GOVERNMENT SUPPORT CLAUSE

This invention was made with government support under Grant Number GM127463 awarded by the National Institutes of Health. The government has certain rights in the invention.

### TECHNICAL FIELD

The present disclosure relates to computational methods of compressing genetic information and, in particular, to computational methods and systems for compressing genetic information in multiple reading frames to reduce the total amount of linear sequence required to encode a set of genetic elements by overlapping the sequences that encode them.

### BACKGROUND

There are many situations where the amount of deoxyribonucleic acid (DNA) or ribonucleic acid (RNA) available to encode genetic information (e.g., protein coding sequences, RNAs, regulatory elements) is limiting. These situations include, but are not limited to, delivery of genetic information (e.g., large genes) to animals (e.g., humans, domestic animals) or plants (e.g., crops) for gene therapy or genetic editing applications using viruses such as adeno-associated virus (AAV) or other vectors (e.g., other viruses, or non-viral delivery methods). Additional situations involve CRISPR-Cas (or similar programmable DNA-binding proteins such as zinc finger nucleases (ZFNs) and transcription activator-like effector nucleases (TALENs)) based systems for genome editing including: targeted DNA cutting, homology directed repair, base editing, transcriptional regulation, translational regulation, or splicing regulation. In some cases there is a strict upper-limit on the amount of DNA or RNA that can be delivered. For example, AAV vectors for mammalian gene delivery are limited to genetic cargos of <5 kb. Geminivirus vectors have similar limitations. Moreover, it is generally preferable to use shorter sequences because many steps of the engineering process, including delivery, are typically more efficient with shorter sequences.

### SUMMARY OF THE DISCLOSURE

To reduce the total amount of linear sequence (DNA, RNA or other medium) required to encode a set of genetic elements, the present disclosure describes a computational method for compressing genetic information by finding a single sequence that mutually encodes two genetic elements in the same stretch of sequence (a “co-encoding”). The co-encoding can, in principle, be in either strand of a double-stranded encoding and the two elements can be encoded in the same or different reading frames if they are proteins. This technique can also be applied to a single genetic element if it is split into two elements (e.g., a protein like galactosidase or cas9 that can be functionally expressed in two fragments). Potential split points can be identified using computational methods or by functional screens. Split proteins may spontaneously assemble after translation. Alternatively, split proteins can be reconstituted by intein-mediated trans-splicing, mRNA trans-splicing, or known protein-protein interaction domains. Split RNAs may spontaneously assemble through base pairing interactions or be reconstituted through RNA trans-splicing.

Because the natural genetic code is redundant (several codons code for the same amino acid) and many amino acids in proteins are readily substituted, functional genetic elements like proteins admit many DNA representations. The disclosed methods encode information about acceptable nucleotide representations of genetic elements (e.g., proteins or functional RNAs) as a directed acyclic graph (DAG) structure. The DAG is generated from multiple data sources, including codon degeneracy, information from multiple sequence alignments (MSAs), and diverse functional screens of mutant elements. Mutations can include single substitutions as well as insertions, deletions and topological rearrangements of the sequence (e.g., circular permutations and other reorderings). DAG representations of two genetic elements can be used to compute viable co-encodings, which are equivalent to partial intersections of the two DAGs. Individual co-encodings are paths through the resulting intersection graph. Many possible overlaps can be tested by changing the relative positions of the two graphs before calculating the partial intersection.

In nearly all cases with viable co-encodings, many viable sequences are found, many more than can be feasibly tested in a laboratory setting. A computational method for evaluating the quality of individual sequences uses information from natural and mutant sequences to quantify the degree to which overlapping variants preserve the sequence characteristics of functional variants. Data for this ranking procedure is drawn from diverse sources including MSAs, protein structure, and high-throughput functional assays of mutant sequences. A score ranking the quality of a co-encoding is computed, which enables prioritization of variants in a “library generation” procedure. A “library” is a suite of putative functional co-encoding sequences to be tested in a laboratory setting. When high-throughput tests (e.g., functional screens and selections in microbes) are available, large co-encoding libraries (e.g., >10⁵ sequence variants) are generated computationally and synthesized for testing. Smaller libraries (e.g., 10-1000 variants) comprising only the top-ranking variants are designed in cases where low-throughput methods (e.g., in-vitro biochemistry) are the only means of testing. Overall, the disclosed methods integrate multiple sources of data through computation to predict functional co-encodings of two (or more) genetic elements (or fragments thereof). These co-encodings are then tested by experimental means to identify those that work best for the desired application.

A method of compressing genetic information in multiple reading frames by intersecting graph representations, includes for a series of first genetic sequences encoding first proteins or nucleic acid sequences, associating a first score with each possible nucleotide or amino acid residue, insertion, and deletion at each position. The method further includes encoding the first genetic sequences in first computer-readable data structures comprising first directed acyclic graphs (DAGs) or a first finite automatons (FAs) such that (i) a plurality of potential genetic sequences for the first proteins or nucleic acid sequences are encoded in the first data structures, (ii) each edge in the first DAGs or first FAs represents a nucleotide residue, insertion, or deletion at that position and the first score associated with the nucleotide residue, insertion, or deletion at that position, (iii) each path through the first DAGs or accepted sequence in the first FAs represents a potential sequence encoding one of the first proteins or nucleic acid sequences, and (iv) for each path through one of the first DAGs or accepted sequence in one of the first FAs, a first aggregate score of the path or accepted sequence is the accumulation of the first score of all edges along the path or accepted sequence. The method includes encoding, in a second DAG or a second FA, overlapping sequences between the encoded first genetic sequences for the first proteins or nucleic acid sequences, calculating, for each edge in the second DAG or the second FA, a second score representing a combined total effect of the component edges of the first data structures, and selecting, according to a second aggregate score of each of the edges, a sequence represented by a path through the second DAG or the second FA.

A system includes a computer processor, and a memory, communicatively coupled to the computer processor. The memory stores instructions, executable by the computer processor to cause the processor to perform a number of steps. The steps include, for a series of first genetic sequences encoding first proteins or nucleic acid sequences, associating a first score with each possible nucleotide or amino acid residue, insertion, and deletion at each position. The steps also include encoding the first genetic sequences in first computer-readable data structures comprising first directed acyclic graphs (DAGs) or first finite automatons (FAs) such that (i) a plurality of potential genetic sequences for the first proteins or nucleic acid sequences are encoded in the first data structures, (ii) each edge in the first DAGs or first FAs represents a nucleotide residue, insertion, or deletion at that position and the first score associated with the nucleotide residue, insertion, or deletion at that position, (iii) each path through the first DAGs or accepted sequence in the first FAs represents a potential sequence encoding one of the first proteins or nucleic acid sequences, and (iv) for each path through one of the first DAGs or accepted sequence in one of the first FAs, a first aggregate score of the path or accepted sequence is the accumulation of the first score of all edges along the path or accepted sequence. The steps further include encoding, in a second DAG or a second FA, overlapping sequences between the encoded first genetic sequences for the first proteins or nucleic acid sequences. Still further, the steps include calculating, for each edge in the second DAG or the second FA, a second score representing a combined total effect of the component edges of the first data structures, and selecting, according to a second aggregate score of each of the edges, a sequence represented by a path through the second DAG or the second FA.

### BRIEF DESCRIPTION OF THE FIGURES

FIG. 1 depicts a conceptual diagram of a computational method according to the present disclosure.

FIG. 2A depicts a functional representation of a particular three-element peptide.

FIG. 2B depicts an alternate representation of the peptide of FIG. 2A.

FIGS. 3A and 3B illustrate, respectively, deterministic finite automaton (DFA) and nondeterministic finite automaton (NFA) graph representations of the data of FIGS. 2A and 2B.

FIG. 4 depicts graph representations of a pair of example genetic elements.

FIG. 5 depicts the discovery of a co-encoding sequence resulting from a one-position offset between the pair of example genetic elements of FIG. 4.

FIG. 6 depicts a graph representation of the co-encoding sequence discovered in FIG. 5.

FIG. 7 is a block diagram of an example computational environment in which the methods of the present description may be implemented.

FIG. 8 is a class architecture diagram corresponding to the computational method compression of genetic information.

FIG. 9 is a flow chart illustrating an example method for performing the computational method.

FIG. 10 is a flow chart illustrating an example method for performing a first portion of the method of FIG. 9.

FIG. 11 is a flow chart illustrating an example method for performing a second portion of the method of FIG. 9.

FIGS. 12A and 12B are a flow chart illustrating an example method for performing a third portion of the method of FIG. 9.

### DETAILED DESCRIPTION

This disclosure describes a computational method of designing co-encoding nucleic acid sequences for reducing the number of bases (DNA or RNA) required to encode larger constructs. The co-encoded sequences can be separate genetic elements or single genetic elements that are split and then reassemble in situ using native interactions, intein mediated trans-splicing, mRNA trans splicing, or known/engineered interaction domains.

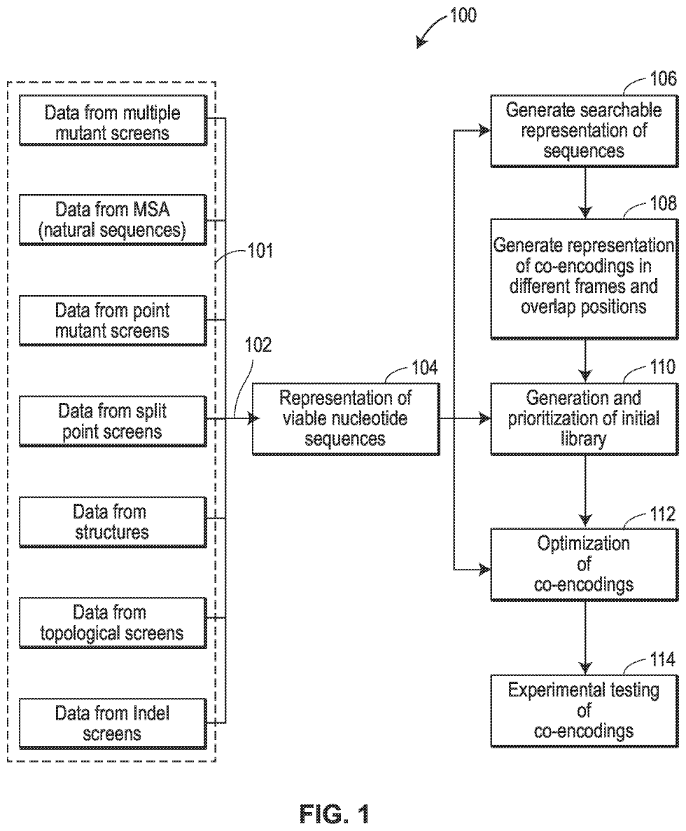

FIG. 1 depicts, at a high level, the computational method 100. The method 100 assembles, from a variety of data sources 101, admissible encodings of relevant genetic elements (block 104). The data sources 101 may include, by way of example and not limitation, data from: multiple mutant screens; multiple sequence alignments (MSAs); point mutant screens; split point screens; three-dimensional structural data of RNAs or proteins; topological screens, and indel (insertion and/or deletion) screens. Functional screens test which sequence variations preserve, enhance, or diminish the function of a protein or amino acid. The various sequence variations include substitutions (i.e., inclusion of one nucleic acid residue for another), insertions, or deletions. Screens can also be carried out on elements with multiple mutations (e.g., multiple substitutions, deletions, or insertions) to evaluate interactions between sources of sequence variation at different positions in the genetic element. For example, both adjacent and non-adjacent substitutions, insertions, and/or deletions may be evaluated to determine the effects of these variations on the overall structure of the genetic element in question.

The admissible encodings of relevant genetic elements may be determined for a number of nucleic acids, proteins, or other genetic elements/sequences and stored, for example, in a library of such admissible encodings. Alternatively, a user may indicate or select specific genetic elements or sequences of interest, and the admissible encodings of the selected genetic elements or sequences may be determined from the data sources 101 according to the selection.

In any event, for each of the genetic elements, a score is associated with each possible residue, insertion, and deletion at each position in the genetic element. In various embodiments, the scores reflect different types of metrics, depending on the particular application, payload genetic elements, and/or goals. In some embodiments, the score reflects a likelihood or statistical probability of a residue, insertion, or deletion at a position, while in other embodiments, the score may reflect a fitness metric (e.g., fitness of the resulting genetic element for performing its intended function). Generally, the score reflects an expression of a probability or effect of the residue, insertion, or deletion at the position in question.

In some embodiments, the type of score employed is determined by the genetic elements under analysis. In other embodiments, the type of score employed may be selected by a user along with the selection of the genetic elements to analyze. In some embodiments, the library of admissible encodings may store multiple types of scores for each residue, insertion, or deletion at each position while, in other embodiments, the scores may be determined or calculated for each genetic element by referencing specific relevant ones of the data sources 101 to calculate the type of score requested or required.

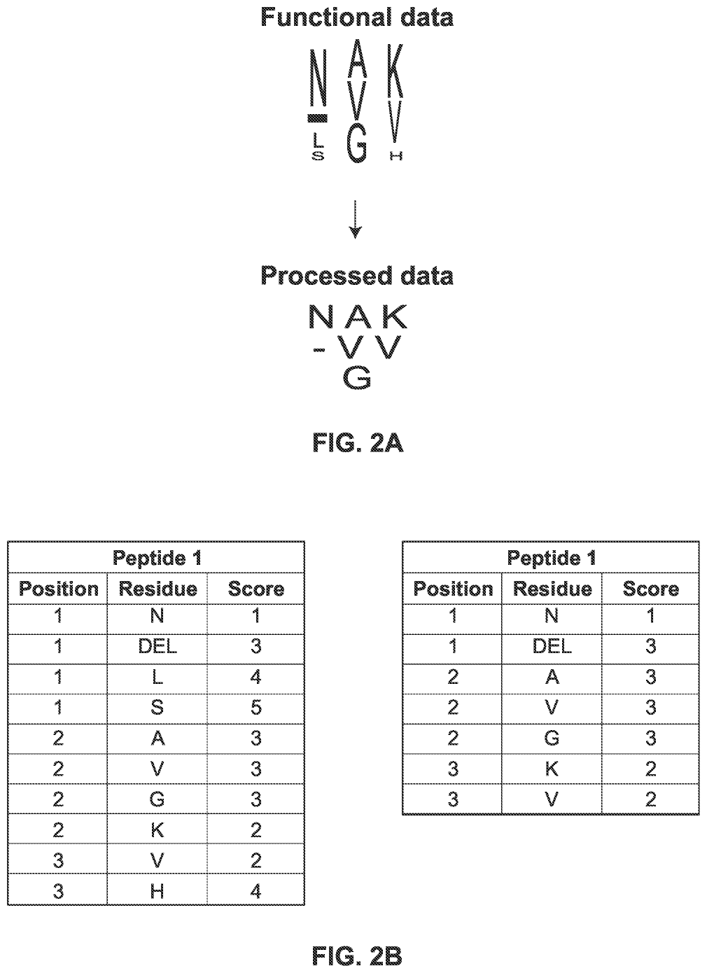

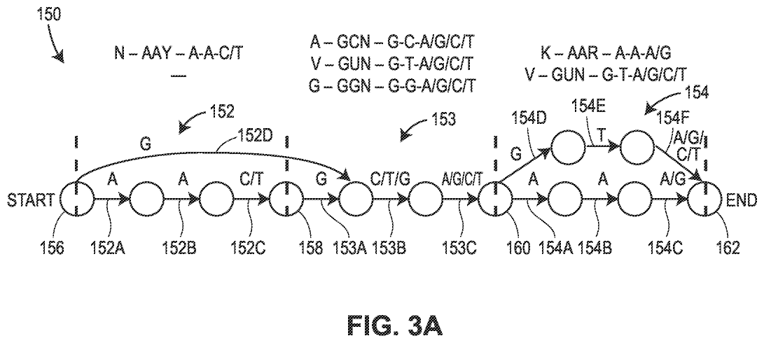

The scores associated with the various substitutions, insertions, and deletions (referred to sometimes as “mutations” for brevity), are used to determine, for each genetic element under evaluation, which sequences for the genetic element are viable. The viable nucleotide sequences are represented in any desired format. FIG. 2A depicts example formats in which viable sequences for a genetic element may be encoded, for instance using letter height to indicate a score associated with a particular residue. In FIG. 2A, a functional representation shows a particular three-element (e.g., three amino acid) peptide has a high score associated Asparagine (N) in a first residue position, but may also have a deletion at this residue position or, with much lower score, Leucine (L) or Serine (S); may have in a second residue position any of Alanine (A), Valine (V), or Glycine (G) with approximately equal score; and may have in a third residue position either Lysine (K) or Valine (V) with approximately equal score or potentially Histidine (H) with a lower score. FIG. 2A also depicts processed representations of the data, in which only mutations having a score above a pre-selected threshold are included in the processed data. FIG. 2A does not depict the absolute scores as being associated with the mutations, but it should be understood that the scores are maintained and stored.

FIG. 2B shows an alternate format in which such a representation could be stored, specifically, in table form. In FIG. 2B, a first table stores, for each position, the possible residues at that position and the scores associated with the residues at that position. A second table stores the processed form of that table, in which residues with scores falling below a certain threshold (scores >3, in this example) are removed.

Referring again to FIG. 1, a searchable representation of each of the genetic elements is created from the representations of the viable sequences (block 106). In embodiments, the viable sequences are converted to a directed acyclic graph (DAG) or a finite automaton (FA) such as a nondeterministic FA. The DAG and FA are isomorphic to one another. That is, mapping from one to the other is reversible. The DAG or FA is built such that a path through the DAG, or an accepted sequence in the FA, represents a potential sequence that encodes the desired genetic element. Each edge or connection in the DAG or FA is associated with the corresponding score for the element encoded by the edge or connection, and the aggregate score of the sequence is the accumulation of the scores of all edges along the path or accepted sequence.

In order to encode information about allowed sequences in a DAG format, the incorporation of a particular nucleotide at a position in the sequence is represented as an edge in the graph. This edge has an associated nucleotide (or degenerate code indicating several possible nucleotides) and a length defined according to one of the variety of scoring metrics such as probability, negative log-likelihood, or fitness effect. In this formulation, multiple edges leaving any one node may be associated with the same nucleotide, this results in a DAG that is isomorphic to a nondeterministic FA. Alternatively, this graph could be arranged such that each node could have at most one outgoing edge for each nucleotide in which case the graph is isomorphic to a deterministic FA. In a graph constructed in this manner, a node with no incident edges represents the starting position. A node with no outgoing edges represents an accepting state or end state. In this construction, any path from the start position to the end position represents a potential sequence, and the length of this path represents that sequence's score. This construction allows the storage of a large number of sequences in minimal space by storing the rules of how to make and score sequences instead of the sequences themselves. This avoids the combinatorial (O(n!)) increase in the number of sequences that would have to be stored. At the same time, this allows fast longest/shortest pathfinding algorithms to generate best scoring sequences from the graph very quickly. Further, modifying edge lengths according to desirable characteristics of the sequences such as GC content, codon usage, proximity to known functional sequences, position entropy, or random variation, run linearly with respect to edge number. This then allows new paths to be generated that are weighted according to new criteria.

A similar construction can be used in order to represent sequence overlaps. Edges in the DAG represent a nucleotide (or degenerate multi-nucleotide) that can be accepted by both sequences, and the length of the edge represents the combination of the scores (e.g. product for probabilities, or sum for negative log likelihoods). This configuration benefits from all of the advantages mentioned above. However, paths through this graph represent sequences that contain overlaps of both parental sequences and scores that represent the joint score of the overlap. To generate this overlap graph representation, two sequence DAGs can be compared using graph search algorithms given an overlap start position in each sequence. This search adds edges that are valid combinations of edges in the parental graph, and trims paths that end at nodes which are not end nodes but have no valid outgoing paths. This removes complexity from the graph. This graph can then be used to generate sequences as described for single sequence graphs and can be modified to reflect different sequence priorities as described there. By generating sequences that reflect design priorities or high scoring sequences, sequences likely to perform well in downstream steps can be efficiently sampled from the combinatorially large (and therefore computationally intractable) set of possible sequences that could be made for a particular overlap.

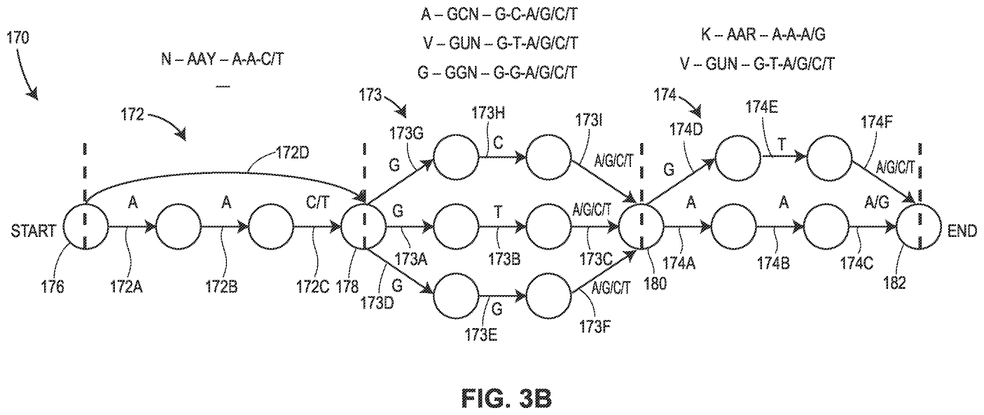

FIGS. 3A and 3B illustrate graph representations 150 and 170 of the data of FIGS. 2A and 2B. In the graph representations 150 and 170, each of three amino acid residue positions 152-154 is depicted. In the graph 150, the graph representation is isomorphic to a DFA, while in the graph 170, the graph representation is isomorphic to an NFA. Important characteristics marking that the graph 150 represents a DFA include that no edges are unlabeled with a nucleotide residue, and that there is never more than one edge leaving a single node that can allow the same nucleotide. The graph 170 conversely contains an edge 172D that is not labeled with a nucleotide, and edges 173A, 173D, and 173G that all leave the node 178 but all allow G nucleotides. These characteristics indicate that the graph 170 cannot be a DFA and in fact represents an NFA. In both FIG. 3A and FIG. 3B, some edges are labeled with multiple nucleotide options. These are drawn to indicate where multiple nucleotides can be accepted starting at this starting node and ending at this ending node. In instantiations of the method, this concept could be represented by one edge that accepts any of the possible nucleotides and has a single score associated with it, or it may be represented as a different edge for each nucleotide that may have a different score.

Turning to the graph 150, the DFA graph representation is discussed in more detail. In the first amino acid residue position 152, either an N (Asparagine) or a deletion is viable in that position. In the second amino acid residue position 153, any of an A (Alanine), a V (Valine), or a G (Glycine) is viable in that position. In the third amino acid residue position 154, either a K (Lysine) or a V (Valine) is viable in that position. Within the graph 150, each of the amino acid residue positions 152-154 is represented by nodes (represented by circles) and edges (represented by direction specific lines). In the amino acid residue position 152, a start node 156 denotes the start of the peptide. An Asparagine amino acid is encoded by a sequence AAY (using standard genetic coding), in which the Y denotes a wobble for which either a C or T nucleotide may be present. Thus, the Asparagine amino acid may be encoded either by the sequence A-A-C or by the sequence A-A-T. As a result, the graph representation 150 depicts a first edge 152A associated with a nucleotide A, a second edge 1528, associated with a nucleotide A, and a third edge 152C associated with either a nucleotide C or a nucleotide T. Edge 152C could also be represented as a separate edge for each nucleotide in particular instantiations. Each of the edges 152A-C is separated from the others by a node, and each of the edges 152A-C is associated with a corresponding score for the corresponding nucleotide. The nucleotide score may be derived from the score for the relevant amino acid or amino acids. The amino acid residue position 152 also depicts an edge 152D from the start node 156 to a node between the edges 153A and 153B in the next amino acid position 153, indicating that a deletion is a viable option at the first amino acid residue position 152, and has associated with it a corresponding score for the deletion and a G nucleotide representing the first nucleotide of the amino acid position 153. Edges associated with deletions must still be associated with a symbol for the graph to remain isomorphic to a DFA.

Similarly, in the amino acid residue position 153, the node 158 separates the first and second amino acid residue positions 152 and 153. The amino acids Alanine, Valine, and Glycine are notated, respectively, as GCN, GUN, and GGN, with N denoting a wobble for which any nucleotide may be present. Thus, each potential amino acid at the second residue position 153 has a first edge 153A associated with a G nucleotide, and a third edge 153C associated with any one of an A, C, T, or G, nucleotide. The edge 153C could also be represented as a separate edge for each nucleotide in particular instantiations. A second edge 153B— representing the second nucleotide encoding of the codon—is associated with a C, T, or G, nucleotide, depending on whether the second amino acid residue is Alanine, Valine, or Glycine. The edge 153B could also be represented with a separate edge for each nucleotide in particular instantiations. Each of the edges 153A-C is separated from the others by a node, and each of the edges 153A-C is associated with a corresponding score for the corresponding nucleotide.

Likewise, in the amino acid residue position 154, a node 160 separates the second and third amino acid reside positions 153 and 154. The amino acids Lysine and Valine are notated, respectively, as AAR or GUN, with R denoting a wobble for which either an A or a G nucleotide may be present. A first path from the node 160 to an end node 162 denoting the end of the peptide sequence includes edges 154A-C and represents the codon for the Lysine amino acid, while a second path from the node 160 to the node 162 includes edges 154D-F and represents the codon for the Valine amino acid. The edges 154A-C are associated, respectively, with nucleotides A, A, and either A or G, and respective scores associated with the corresponding nucleotides. Similarly, the edges 154D-F are associated, respectively, with nucleotides G, T, and any one of A,C, G, and T, and respective scores associated with the corresponding nucleotides. Any of the edges 154C and 154F could also be represented as a separate edge for each nucleotide in particular instantiations.

We will now discuss the NFA representation graph 170 in more detail. FIG. 3B displays one possible way to draw a DAG isomorphic to a NFA that encodes the correct sequence. Other arrangements are possible that would still be DAGs and would remain isomorphic to a NFA. These changes can include but are not limited to splitting edges that accept multiple nucleotides into separate edges that only accept one nucleotide or consolidating multiple edges and nodes such that the graph still ensures that only the proper amino acids can be encoded. In the first amino acid residue position 172, either an N (Asparagine) or a deletion is viable in that position. In the second amino acid residue position 173, any of an A (Alanine), a V (Valine), or a G (Glycine) is viable in that position. In the third amino acid residue position 174, either a K (Lysine) or a V (Valine) is viable in that position. Within the graph 170, each of the amino acid residue positions 172-174 is represented by nodes (represented by circles) and edges (represented by direction specific lines). In the amino acid residue position 172, a start node 176 denotes the start of the peptide. An Asparagine amino acid is encoded by a sequence AAY (using standard genetic coding), in which the Y denotes a wobble for which either a C or T nucleotide may be present. Thus, the Asparagine amino acid may be encoded either by the sequence A-A-C or by the sequence A-A-T. As a result, the graph representation 170 depicts a first edge 172A associated with a nucleotide A, a second edge 1728, associated with a nucleotide A, and a third edge 172C associated with either a nucleotide C or a nucleotide T. The edge 172C could also be represented as a separate edge for each nucleotide in particular instantiations. Each of the edges 172A-C is separated from the others by a node, and each of the edges 172A-C is associated with a corresponding score for the corresponding nucleotide. The nucleotide score may be derived from the score for the relevant amino acid. The amino acid residue position 172 also depicts an edge 172D from the start node 176 to a node 178 between amino acid positions 172 and 173, indicating that a deletion is a viable option at the first amino acid residue position 172, and has associated with it a corresponding score for the deletion.

Similarly, in the amino acid residue position 173, the node 178 separates the first and second amino acid residue positions 172 and 173. The amino acids Alanine, Valine, and Glycine are notated, respectively, as GCN, GUN, and GGN, with N denoting a wobble for which any nucleotide may be present. Each potential amino acid at the second residue position 173 has its own first edge 173A, D, or G associated with a G nucleotide, and third edge 173C, F or I associated with any one of an A, C, T, or G, nucleotide. Any of the edges 173C, 173F or 1731 could also be represented as a separate edge for each nucleotide in particular instantiations. A second edge 1728, E, or H—representing the second nucleotide encoding of the codon—is associated with a C, T, or G, nucleotide, depending on whether the second amino acid residue is Alanine, Valine, or Glycine. Each of these edges is arranged into paths, one for each amino acid represented. A first path from the node 178 to an end node 180 denoting the end of the codon includes edges 173A-C and represents the codon for the Valine amino acid, while a second path from the node 178 to an end node 180 includes edges 174D-F and represents the codon for the Glycine amino acid, finally a third path from the node 178 to an end node 180 includes edges 174G-I and represents the codon for the Alanine amino acid. Each of the edges 153A-I is separated from its neighbors by a node, and each of the edges 173A-I is associated with a corresponding score for the corresponding nucleotide.

Likewise, in the amino acid residue position 174, a node 180 separates the second and third amino acid reside positions 173 and 174. The amino acids Lysine and Valine are notated, respectively, as AAR or GUN, with R denoting a wobble for which either an A or a G nucleotide may be present. A first path from the node 180 to an end node 182 denoting the end of the peptide sequence includes edges 174A-C and represents the codon for the Lysine amino acid, while a second path from the node 180 to the node 182 includes edges 174D-F and represents the codon for the Valine amino acid. The edges 174A-C are associated, respectively, with nucleotides A, A, and either A or G, and respective scores associated with the corresponding nucleotides. Similarly, the edges 174D-F are associated, respectively, with nucleotides G, T, and any one of A,C, G, and T, and respective scores associated with the corresponding nucleotides. Any of the edges 174C and 174F could also be represented as a separate edge for each nucleotide in particular instantiations.

When attempting to compress the sequence data for two or more genetic elements, the computational method 100 (FIG. 1) generates a representation of co-encodings in different frames and overlap positions (block 108, FIG. 1). For even two genetic elements under analysis, there may be multiple co-encodings, as different co-encodings may be made possible by selecting differing relative starting positions within each of the genetic elements under analysis, and because at each pair of starting positions, if even one viable co-encoding can be found there are likely to be many possible co-encodings that differ because they choose different nucleotides at variable points. Accordingly, for any pair or group of genetic elements, a new DAG or FA is generated such that any path through the new DAG or accepted sequence in the new FA represents an allowed overlap between the starting sequences. The new representation can be created, for example, by performing a graph search on pairs of states, one from each parent data structure (DAG or FA), and adding new child states as pairs of states that are reachable from the parent state in each graph by adding the same residue. This procedure may be performed according to any well performing graph search algorithm such as breadth- or depth-first searches. This procedure avoids explicitly detailing sequences and instead defines a rule for how to find and score such sequences, reducing the complexity of the problem. In embodiments, the final tree can be trimmed by known, efficient backtracking methods to remove any state pairs that do not lead down productive paths. In the new DAG or FA, the weights on the edges are calculated from the edges of the component genetic component DAGs such that they properly represent the combined total effect of having taken a path incorporating the associated edges.

A library of co-encodings may be created (block 110). The library may be prioritized according to various characteristics such as co-encoding length, total payload size, suitability for the intended purpose, and the like. A set of top co-encodings may be selected according to the prioritization and/or the scores associated with each co-encoding before further optimization is performed. These scores can be adjusted by taking into account non-local interactions. The library of co-encodings may be optimized further (block 112) by adjusting the sequence to maximize positive non-local interactions and minimize negative non-local interactions. Experimental (e.g., in vitro, in vivo, in silico, etc.) testing may be conducted (block 114) on selected and/or optimized candidate co-encodings.

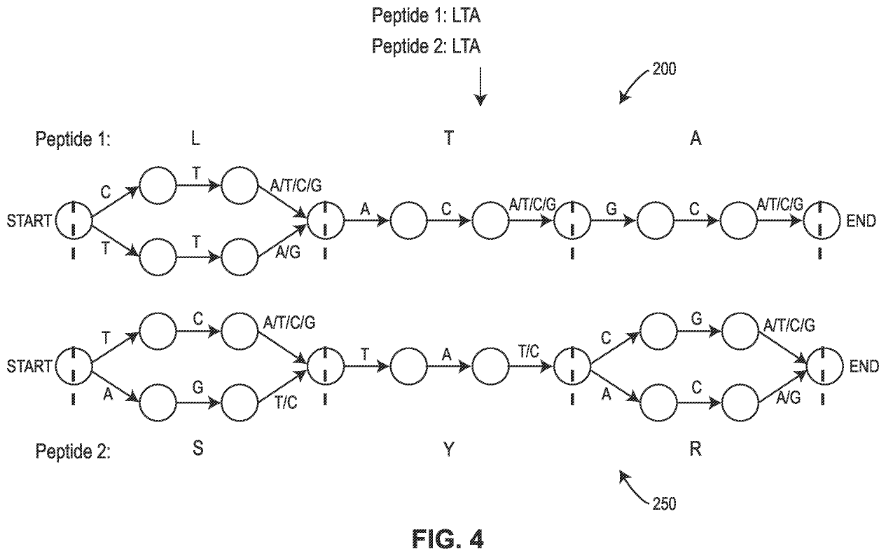

The general concept of the generation of co-encoding sequences will be illustrated further with reference to FIGS. 4-6. FIG. 4 shows two graph representations of example genetic elements 200 and 250, peptides, each comprising three amino acids. The example genetic element 200 comprises Leucine (L), Threonine (T), and Alanine (A) (LTA), while the example genetic element 250 comprises Serine (S), Tyrosine (Y), and Arginine (R) (SYR). The graph representation of the peptide 200 encodes that Leucine is encoded by the codons CTA, CTT, CTC, CTG, TTA, or TTG; that Threonine is encoded by the codons ACA, ACT, ACC, or ACG; and that Alanine is encoded by the codons GCA, GCT, GCC, or GCG. Similarly, the graph representation of the peptide 250 encodes that Serine is encoded by the codons TCA, TCT, TCC, TCG, AGT, OR AGC; that Tyrosine is encoded by the codons TAT or TAC; and that Arginine is encoded by the codons CGA, CGT, CGC, CGG, ACA, or ACG. The available nucleotides at each position are as depicted in Table 1, below.

TABLE 1

| Peptide | C | T | A/T/C/G | A | C | A/T/C/G | G | C | A/T/C/G |
|---|---|---|---|---|---|---|---|---|---|
| 1 | T | T | A/G | | | | | | |
| Peptide | T | C | A/T/C/G | T | A | T/C | C | G | A/T/C/G |
| 2 | A | G | T/C | | | | A | C | A/G |

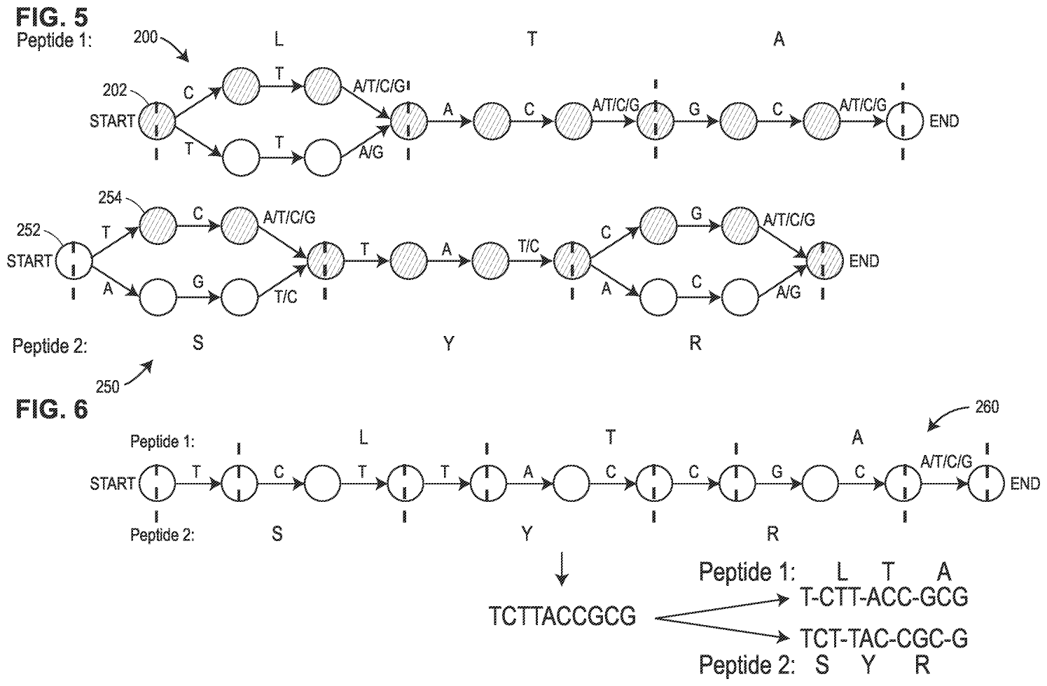

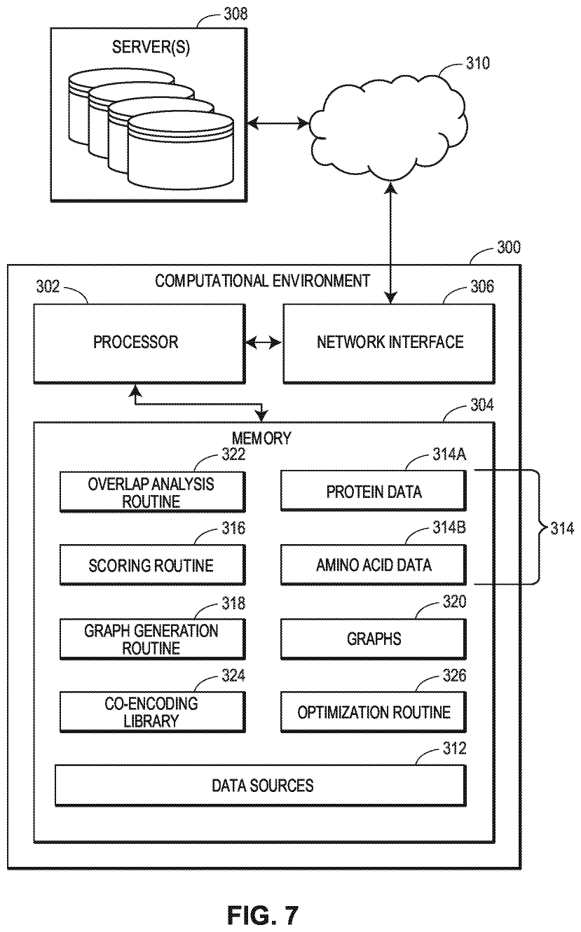

FIG. 5 illustrates that by offsetting the start positions of the peptide 200 and the peptide 250 by one nucleotide residue, the peptides 200 and 250 may be co-encoded. That is, by setting a start node 202 of the peptide 200 at a position offset from a start node 252 of the peptide 250 and, specifically, at the position of a second node 254 of the peptide 250, a co-encoding sequence may be generated. As can be seen in Table 2, below, relative to the node 202 of the peptide 200, one path through the DAG depicted includes the sequence C-T-T-A-C-C-G-C-A/T/C/G, while relative to the node 252 of the peptide 250, one path through the DAG depicted includes the sequence T-C-T-T-A-C-C-G-C. Offset by one node, as depicted in Table 2, the sequence T-C-T-T-A-C-C-G-C-A/T/C/G would encode both peptides 200 and 250, as illustrated in FIG. 6. FIG. 6 shows a co-encoding graph 260 for the co-encoding sequence depicted in FIG. 5.

TABLE 2

| Peptide | | C | T | A/T/C/G | A | C | A/T/C/G | G | C | A/T/C/G |
|---|---|---|---|---|---|---|---|---|---|---|
| 1 | | T | T | A/G | | | | | | |
| Peptide | T | C | A/T/C/G | T | A | T/C | C | G | A/T/C/G | |
| 2 | A | G | T/C | | | | A | C | A/G | |

Once a co-encoding sequence is encoded in a graph, the graph can be used to generate potential overlap sequences by using any of a variety of longest/shortest path algorithms, stochastic algorithms, deterministic algorithms, or a combination, coupled with adjustments of edge weights to up or down weight paths with specific attributes. These algorithms can be employed to quickly generate a large number of potentially overlapping sequences with associated scores that are maximized for attributes of their sequence, such as highest or lowest scores, similarity to a specific sequence, amino acid preference, or codon usage. Potential overlapping sequences can then be further scored while taking into account interactions between distant positions to account for any non-local effects that are expected from mutagenesis or bioinformatics studies.

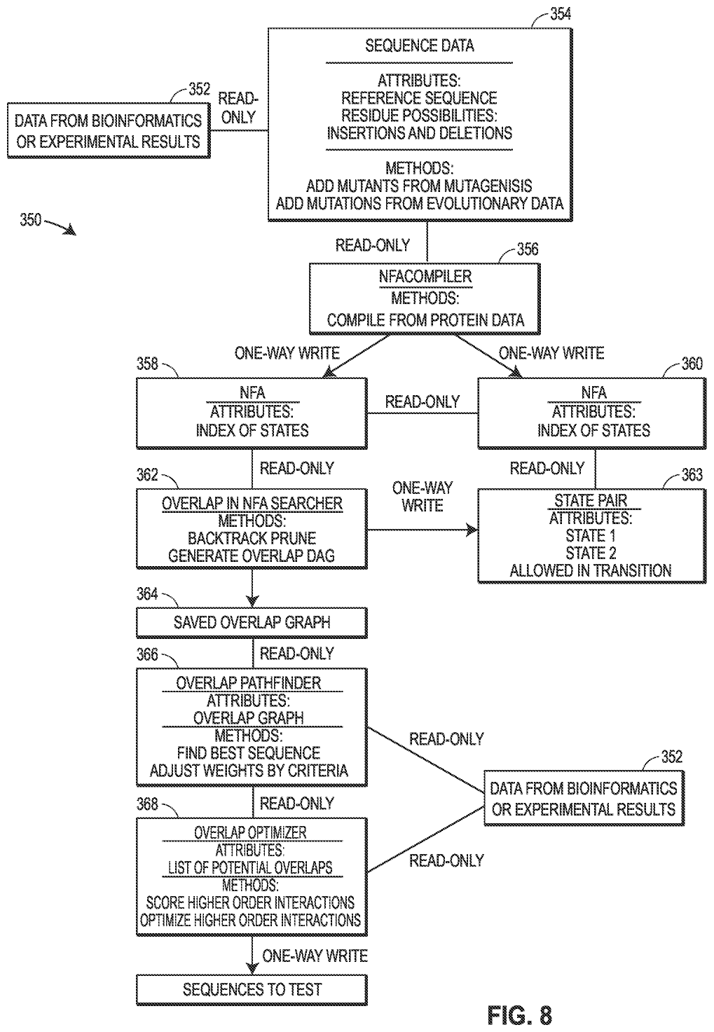

The computational methods described herein may be implemented in a computer environment. An example computational environment 300 is depicted in FIG. 7. The computational environment 300 may be a physical workstation or virtual (e.g., cloud-based) computing environment executing on a computing platform such as Amazon Web Services or Microsoft Azure. In any event, the computational environment 300 includes a processor or processors 302 coupled to a memory 304. In embodiments, a network interface 306 may couple the computational environment 300 to one or more servers and/or databases 308 via a network 310, such as the Internet. The computational environment 300 executes a variety of routines as described herein that, collectively, perform the methods described herein. The memory 304 stores various routines that perform methods, or portions thereof, for associating scores with various mutations, encoding genetic elements in data structures, determining overlap between the data structures, generating data structures to represent the determined overlap, optimizing overlap sequences, etc. It should be understood that while depicted as stored in the memory 304, the routines may be executed by the processor 302 and may cause the processor to retrieve data from the memory 304, read instructions from the memory 304, and/or store results (i.e., output data) in the memory 304. To the extent that the present description refers to routines as “doing” something, it will be understood that the routines are actually causing the processor to perform certain actions.

Specifically, the memory 304 may store a variety of data sources 312 corresponding to some or all of the data sources 101 described above with respect to FIG. 1. The data sources 312 may be periodically updated, for example, via the network 310 from the databases and/or servers 308. In embodiments, the data sources 312 are not stored locally in the memory 304, but are accessed, when needed, directly from the servers and/or databases 308 via the network 310.

The memory 304 may also store genetic element data 314. The genetic element data 314 may include various genetic elements that may be selected for analysis and/or compression using the methods described herein. For example, the genetic element data 314 may include protein data 314A for a variety of proteins, such that a user could select two or more of the proteins to determine whether suitable overlapping sequences exist for the selected proteins, which would allow for compression. The protein data 314A may include, for example, for each protein, the possible amino acid residue sequences that make up the protein. At the same time, a set of amino acid data 314B may include, for each of the amino acids the possible nucleic acid residue sequences that code for the particular amino acid.

Of course, each amino acid, protein, or other genetic element may be susceptible to any number of nucleic acid residue substitutions, insertions, or deletions. That is, for a given nucleotide sequence, a substitution, insertion, or deletion may occur at any position, with a potentially known probability and, potentially, a known effect on the overall functionality or suitability of the resulting nucleotide sequence. The data sources 312 may include data directed to the probability of a particular substitution, insertion, or deletion at a specific position, may include data directed to the advantageous or deleterious effects of such a substitution, insertion, or deletion at a specific position, and may provide other data that may be used to develop a score associated with the presence (or absence) of a particular nucleotide at a specific position.

A scoring routine 316 may be stored in the memory and executed by the processor 302 to determine, for a selected genetic element, a score associated with each substitution, insertion, or deletion at each position in the nucleotide or amino acid sequence for the genetic element. The scoring routine may make use of the data sources 312. The scoring routine 316 may store in the memory, the scores associated with each position in the sequence, for each mutation. In embodiments, the type of scoring to be used may be selected by the user, while in other embodiments the type of scoring used may be determined according to the genetic element type (e.g., protein, gene, etc.) or according to the intended use of the co-encoding sequence (e.g., gene editing, etc.).

A graph generation routine 318 may use the genetic element data 314, the data sources 312, and the output of the scoring routine 316 to generate data structures (e.g., FAs or DAGs) representing each of the selected genetic elements. The resulting data structure for each selected genetic element may include the information to generate a representation of every possible sequence of nucleotide residues, along with the scores for each potential substitution, insertion, or deletion at each position. In embodiments, the graph generation routine may ignore potential substitutions, insertions, or deletions having scores that are above (or below) some predefined threshold, such as those that are exceedingly improbable, unsuitable, or undesirable. The resulting data structures may be stored (e.g., in graph storage 320) in the memory 304.

An overlap analysis routine 322 may retrieve data from the graph storage 320 and may analyze graphs for selected genetic elements to determine overlapping sequences between the selected genetic elements. The overlap analysis routine 322 may analyze the graphs for the selected elements by shifting the starting points of each genetic element relative to the other(s) to determine whether there may be overlapable segments. In embodiments, the overlap analysis routine 322 may also analyze the reverse complement of one or more of the selected genetic elements—for example, comparing the reverse complement of a first genetic element relative to a second genetic element. The overlap analysis routine 322 may also generate a new graph (or FA) data structure representing the overlap sequences between the selected genetic elements, and may associate with each edge in the graph or FA an aggregate score representing the combined effects of the corresponding edges in the graphs for the selected genetic elements. The data structure representing the overlap sequences may likewise be stored in the memory 304 (e.g., in the graph storage 320).

In embodiments, the overlap analysis routine 322 (or another routine) may also generate, from the overlap data structure, a co-encoding library 324. The overlap analysis routine 322 (or other routine) may traverse the various paths or acceptable states through the overlap data structure to determine nucleotide sequences, exhibiting various levels of overlap, that encode the selected genetic elements. Each of the sequences in the co-encoding library 324 may have associated with it one or more scores. For example, each sequence in the co-encoding library 324 may have associated with it a score for each of the selected genetic elements encoded by the co-encoding sequence, and/or may have associated with it an overall score indicative of the relative suitability of the co-encoding sequence.

An optimization routine 326 may further score and/or optimize the sequences in the co-encoding library 324 using, for example, data from bioinformatics studies or experimental results (e.g., data from the data sources 312) to inform knowledge of higher order interactions between nucleotides at various positions. The best scoring co-encoding sequences may then be selected for in vivo, in vitro, and/or in silico testing.

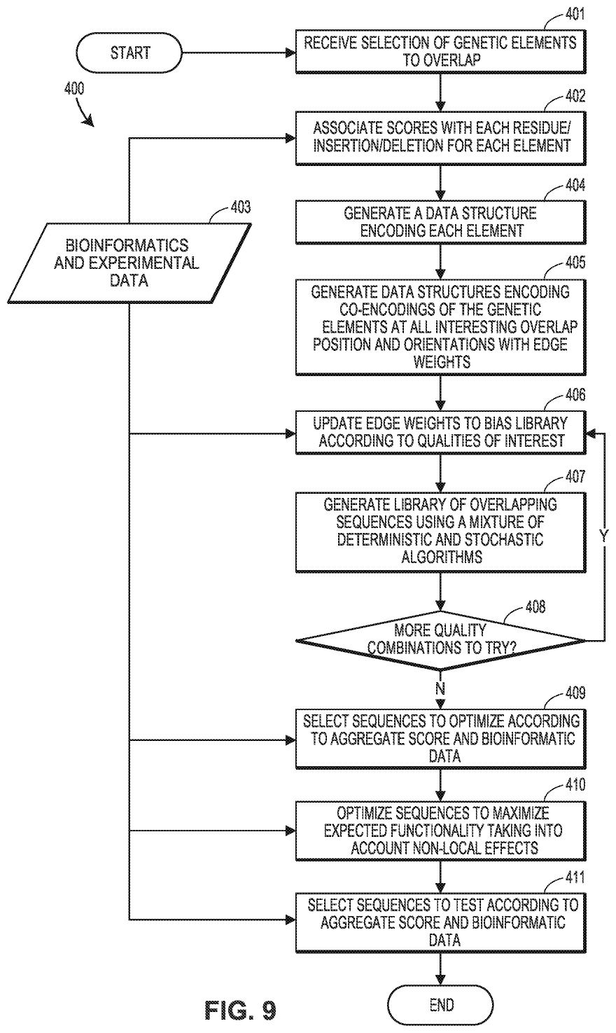

FIG. 8 is a class architecture diagram 350 corresponding to the computational method 100 compression of genetic information. Data 352 from bioinformatics and experimental results (e.g., the data from the data sources 312 corresponding to the data sources 101 of FIG. 1) are collated in a SequenceData class 354 that uses methods for adding mutants from mutagenesis and adding mutations from evolutionary data. That is, the methods of the SequenceData class use the data 352 to determine for a genetic element which residues, substitutions, insertions, and deletions are possible at each position in the genetic element. An NFACompiler class 356 reads the data compiled by the SequenceData class 354 and uses the data to create the FA or DAG from the protein data. The output of the NFACompiler class results in data structures 358 (FA or DAG) and state information 360 indicating allowed transitions. A class 362 reads the data structures 358 and, using the methods thereof generates and prunes an overlap data structure 364, creating state pair objects 363 to hold positions in the parent data structures 358. A class 366 reads the overlap data structure 364, adjusts edge weights according to criteria from the experimental and bioinformatics data 352 or intrinsic characteristics such as codon choice, and determines potential co-encoding sequences. An optimizer class 368 uses the potential co-encoding sequences and the experimental and bioinformatics data 352 to score and optimize the co-encoding sequences, and to adjust co-encoding scores using knowledge of higher order interactions, resulting in a number of co-encoding sequences for testing.

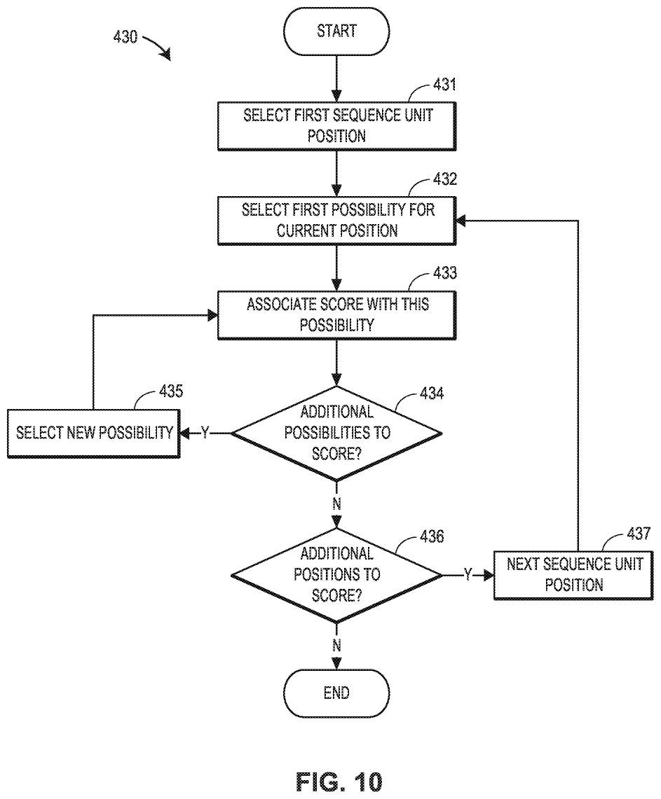

FIG. 9 is a flow chart illustrating an example method 400 for performing the computational method described herein. In the example method 400, we do not impose any limits on the number of sequences to be overlapped, but in practice these should be at least 2 (though this could include one genetic element that is passed in twice or split into two so it can be overlapped with itself) and of course every additional sequence will increase the constraints on the system. The method 400 begins with the receipt of a selection of at least two genetic elements (such as first and second genetic elements) on which to perform the method (block 401). As should by now be understood, the received selection of genetic elements may be a selection of genetic elements including genes, proteins, amino acids, or any sequence of nucleotide residues. The selected genetic elements can be from the same category of elements (e.g., two RNAs, two proteins, etc.) or from different categories of elements (e.g., a RNA and a protein).

For each genetic element, a score is associated with each nucleic acid residue, amino acid residue, insertion, or deletion at each position (block 402). The data for determining the scores associated with each nucleic acid residue, insertion, or deletion at each position of the first sequence is taken from bioinformatics and experimental data 403 (which may correspond to the data sources 101 described with respect to FIG. 1). The genetic elements are encoded as data structures (block 404) and, in particular, as a DAG or FA, that includes, for each position in the nucleotide sequence, edges corresponding to possible nucleotide residues, insertions, and deletions possible at that position. Each edge in any of the data structures has, associated with it, the score for the corresponding nucleotide residue, insertion, or deletion at that position, such that each path or accepted sequence through the data structure corresponds to a potential sequence of nucleotide residues encoding the first genetic element and has an associated score that, as an aggregate of the scores of the edges associated with the path or accepted sequence, represents a score for that path or accepted sequence. The scores for these edges can be the exact nucleotide scores associated in block 402, or they may be scores derived from those in 402. An instance of this may be if the scores in 402 are associated with amino acids not nucleotides, in this case, you may want the score of the first committed edge into the codon to hold the entire score and the other edges to hold placeholder scores such as a probability of 1, alternatively, you may desire to divide the amino acid score among the nucleotides in the codon, or place the full score at each position. Notably, there may be multiple edges at any node corresponding to the same nucleotide, and this can be used to denote a variety of important details that change the score associated with the edge and/or downstream paths that can be followed from that edge. As a non-exclusive example, different edges for the same nucleotide could indicate paths representing different amino acids even though these amino acids have codons with the same nucleotide at that position, this allows the different edges to contain the proper score and ensure that from each edge only downstream edges belonging to the same codon can be reached. It is also important to note that there are likely to be multiple nodes at each position in the nucleotide sequence, and that some of these nodes may not be accessible from all upstream nodes at earlier positions in the protein and may not contain edges to the same downstream nodes. In embodiments, the first data structure may be pruned to include only edges with an associated score that meets certain criteria (e.g., above or below a particular threshold such as a probability threshold, or a functionality threshold

Once the data structures have been created, encoding, the potential nucleotide sequences for the selected genetic elements, the method 400 includes encoding, in one or more co-encoding data structures and, particularly, one or more DAGs or FAs, overlapping sequences between genetic element data structures such that each co-encoding data structure captures a particular position and orientation of the relevant genetic elements, and all interesting positions and orientations are accounted for (block 405). Each edge in a co-encoding data structure corresponds to a combination of edges in the genetic element data structures that are reached at the same point in the progression through an overlapping path in the overlapped data structures and associated with overlapping sets of nucleotides. Accordingly, the score for each edge of the co-encoding data structure is the aggregation of the scores for the corresponding edges of the overlapping genetic element data structures data structures. Similarly, the associated nucleotide or nucleotides is the intersection of the sets of the associated nucleotide(s) of the overlapped edges in the genetic element data structures. In embodiments, the scores may be updated or adjusted according to the bioinformatics and experimental data 403. Further, because shifting the relative start positions for the genetic element data structures, or analyzing the reverse complement of data structures with respect to various start positions of the other data structures, may result in different sets of overlapping sequences, a number of co-encoding data structures may be generated, with each corresponding a different relative start position between the genetic element data structures.

As a result, when block 405 is executed it may create multiple new co-encoding data structures, with each new co-encoding data structure corresponding to the overlapping sequences between the genetic element data structures when the start nodes are shifted relative to one another and/or when a different set of genetic element data structures are analyzed as reverse complements. Thus the number of possible co-encoding positions and orientations and therefore, the number of co-encoding data structures will fall between hard upper and lower bounds. The lower bound is two times the summation of the length of the longest sequence minus the length of the current sequence for each sequence (2 Σᵢ₌₀ⁿ (max(lengths)−lengthsᵢ) for n sequences with lengths in array lengths). The upper bound is calculated similarly but substituting the sum of all the sequence lengths for the longest length (2 Σᵢ₌₀ⁿ (E(lengths)−lengthsᵢ) for n sequences with lengths in array lengths). In practice, however, many of the relative start positions would not be worth analyzing, because the opportunity for compression is not meaningful—for example, when two genetic elements are being overlapped, but the start position of one genetic element is analyzed with respect to only the last few positions of the other genetic element.

The weights of the co-encoding data structure can then be updated to allow for biasing produced sequences towards particular characteristics such as, GC content, codon usage, amino acid usage, or biasing towards specific sequences (block 406). Then, a library of overlapping, co-encoding sequences can be created (block 407), for example by using a shortest/longest path algorithm to select the best scoring co-encoding sequence, by using a partially stochastic algorithm to find sequences similar but distinct from the best scoring sequence, or by using a weighted stochastic algorithm to generate random sequences that prefer high scoring paths. Each sequence in the library of co-encoding sequences may be a sequence that co-encodes the entirety or some part of both of the selected genetic elements (e.g., such as that depicted in FIG. 6), and each may have an associated aggregate score indicating the suitability, probability, or desirability of the co-encoding sequence. Additional sequences may be added to the library by repeating the steps of blocks 406 and 407 using different biasing factors (block 408). Sequences in this library may then be prioritized and selected for further optimization according to their aggregate score and by rescoring them using the bioinformatics and experimental data 405 to account for higher order interactions (block 409). The sequences in the co-encoding library may be optimized (block 410) using the bioinformatics and experimental data 405 to account for higher order interactions, such that sequences may be modified to increase their predicted fitness, this may include removing unfavorable interactions, adding favorable interactions, or adjusting the sequence to improve the score according to a scoring algorithm (block 410). The optimized sequences may then be prioritized and selected for further testing according to a score derived from bioinformatics and experimental data 405 to account for higher order interactions (block 411).

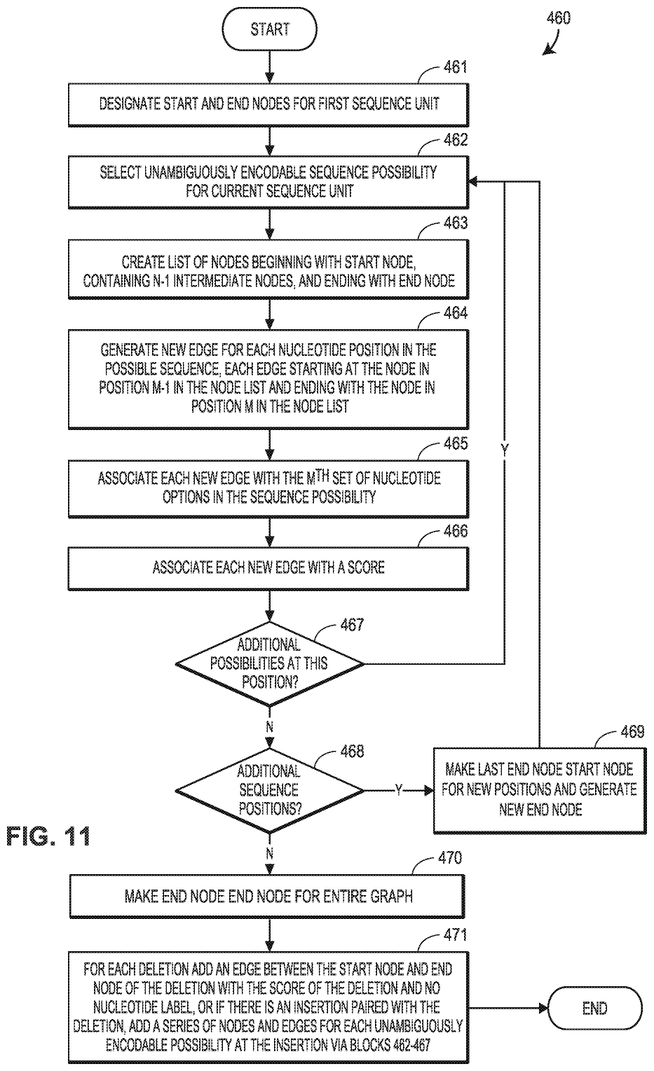

FIG. 10 depicts an example method 430 for associating scores with each residue, insertion, and deletion at each position of sequence (e.g., a method for block 402 of FIG. 9). In the method 430, the sequence to be scored is scored position by position. These positions represent sequence units that can represent individual nucleotide positions, amino acid positions, or other units of sequence. The method 430 starts at a position (block 431), and a possibility that can fill this position in the sequence (block 432). This possibility could include different nucleotides, different amino acids, insertions of various lengths, or deletions starting or ending at this position. Method 430 continues by associating scores with the selected possibility (block 433) and selecting new possibilities to score until no more possibilities are found at this position (blocks 434 and 435) for each new possibility repeating the procedure from block 433. New positions are then selected until all positions in the sequence have been selected (blocks 436 and 437) and the process is repeated from block 432 until every possibility at every position has been scored.

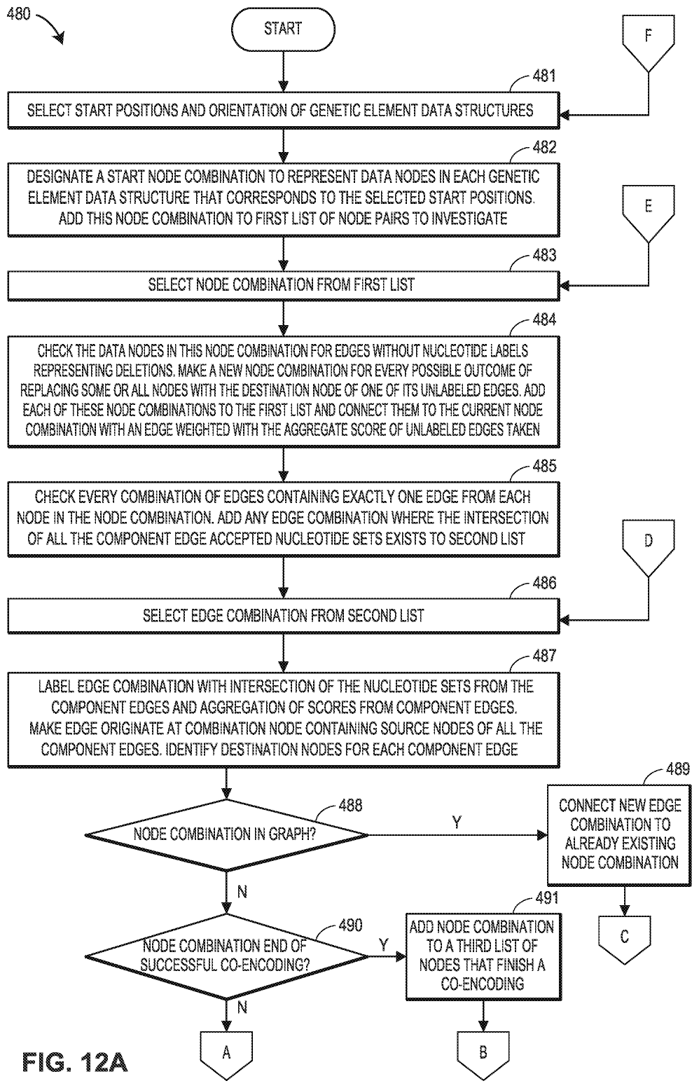

FIG. 11, meanwhile, depicts an example method 460 for encoding a sequence in a data structure (e.g., a method for block 404 of FIG. 9). In the method 460, start and end nodes are designated (block 461). Then an unambiguously encodable unit for the current sequence is selected (block 462) if a nucleic acid sequence is being encoded, this may be a single nucleotide, if a protein is being encoded, this may be an amino acid codon, for some amino acids, this might also be a subset of the possible codons if there are codons that cannot be simplified to an unambiguously encodable representation (e.g. Arginine, Leucine, Serine, and stop codons). It is also possible to define larger units if it is important to define specific sequences that occur together over distances longer than 1 codon. Selecting unambiguously encodable sections is important to ensure that switching doesn't occur mid-section leading to an inadmissible section (e.g. if Serine is the only allowable amino acid at a position, and can be encoded by either of the following unambiguous encodings TCN or AGY it is important to stop switching between encodings halfway through to ensure that you don't for instance produce the codon ACN which encodes Threonine, or TGY which encodes Cysteine just to show two possible switches). It is also not required that all unambiguous encodings have the same length, if for instance one possibility includes an insertion, deletion, or both this is acceptable. Next, in block 463, a list of nodes is created, starting with the designated start node, containing n−1 intermediate nodes, and ending with the designated end node (n here indicates the length of the unambiguous encoding). Then, an edge is created for each nucleotide position connecting the node at the position before the current one to the node at the current position (block 464). Each edge that is added is then associated with the appropriate set of nucleotides (block 465) and score (block 466) for that position in the unambiguous encoding. These scores can be scores calculated for each nucleotide, or they can be scores derived from the score of a larger sequence element such as an amino acid. Scores derived from the scores of larger elements may be assigned by splitting the score between the component edges, assigning every edge the full score, or assigning the full score to the forward direction of the first edge and to the reverse direction of the last edge while filling other edges with scores that do not affect the aggregate score (e.g. probability 1) in order to represent the first committed step into the encoding. This process (blocks 462-466) is then repeated for each unambiguously encodable possibility at the position (block 467). Once every possibility at this position is accounted for, we assess if there are more sequence positions to encode (block 468). If there are more positions to encode, we make the end node, the start node for the new position and generate a new end node (block 469) before repeating blocks 462-469 for every position except the last one, where we repeat blocks 462-468 then progress on to block 470 where we make the current end node the end node of the entire data structure. We can then go back and add deletions that occupy the entirety of a sequence position or cross multiple positions by finding the start and end positions of the deletion in the encoding and adding an edge with no associated nucleotide and a weight associated with the deletion. If however, the deletion is coupled with an insertion, we must add edges and nodes that account for the associated unambiguously encodable sequence possibilities according to the procedure in blocks 462-467 (block 471). When all deletions are added, the encoding is finalized.

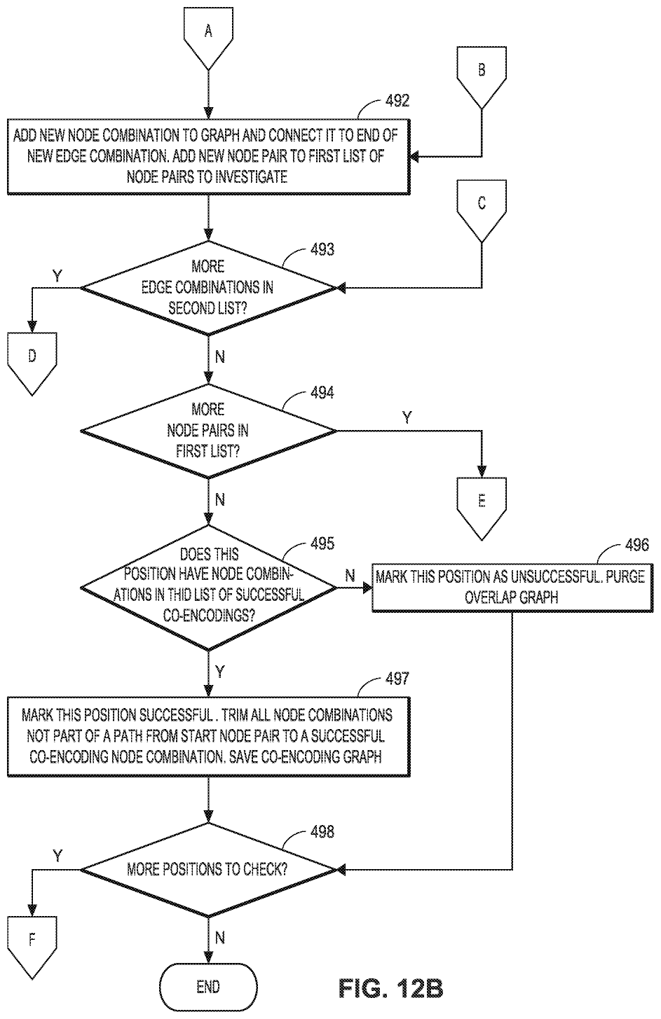

FIGS. 12A and 12B depict a method 480 for encoding, in one or more co-encoding data structures, overlapping sequences between genetic element data structures (e.g., a method for block 405 of FIG. 9). In the method 480, relative start positions and orientations are selected for the genetic element data structures (block 481). In a first pass through the method 480, for example, all the start positions of the genetic element data structures may be aligned. In successive passes through the method 480, different shifts between the start positions of the genetic element data structure start positions may be tried, and different subsets of genetic element data structures may be considered as their reverse complements. A start node of the co-encoding data structure is designated which represents a combination of all the selected start positions of the genetic element data structures, and this node is added to the list of nodes to consider (block 482). A node combination is then selected from the nodes to consider list (block 483). This node combination is then checked for nodes with edges without nucleotide labels representing deletions. A new node combination is then made for every possible outcome of replacing some or all nodes with the destination node of one of its unlabeled edges. Each of these node combinations is then added to the node combinations to consider list and connected to the current node combination with an edge weighted with the aggregate score of unlabeled edges taken (block 484). Then every combination of edges that contains one edge from each component node is checked for edge combinations where the intersection of the nucleotide sets for all component edges is not empty, and these edge combinations are added to a list (block 485). Edge collections are then selected from this list (block 486) and processed. This processing includes labeling the edge combination with the intersection of the nucleotide sets of the component edges and the aggregations of the scores of these edges, as well as setting the origin of the edge collection to the proper node collection (the one representing the origin nodes of all the component edges), and identifying the combination node of all the individual edge destination nodes (block 487). If the new destination node combination is already in the graph (block 488) the edge combination is connected to the previously existing node combination (block 489) and execution continues with block 493. If the destination node combination is not already in the graph however (block 488), a check is done to see if it is the successful conclusion of a co-encoding (block 490), if it is, it is added to a list of co-encoding conclusion nodes (block 491) and either way, the node is added to the co-encoding data structure, added to the list of nodes to investigate and connected to the end of the edge combination (block 492). If there are remaining edge combinations in the list of unprocessed Edge combinations we repeat blocks 486-492 until all edge combinations are processed (block 493). Then blocks 483-493 are repeated until every node combination in the list of unprocessed node combinations has been addressed (block 494). The co-encodings list is then checked for successful co-encodings (block 495) and if there are none, the position is marked unsuccessful and the graph is purged (block 496), but if there are successful co-encodings, the node and edge combinations along paths that lead to successful co-encodings are saved and the position is marked as containing successful overlaps (block 497). Finally blocks 481-497 are repeated until every interesting position and orientation has been considered (block 498).

The co-encodings resulting from the application of one or more of the methods described and claimed herein facilitates the delivery of larger payloads by compressing the data for multiple sequences into a single co-encoded sequence that is shorter than the combined length of the individual sequences. As a result, it may be possible for vectors to carry sequences that would otherwise exceed the maximum payload for the vector, in turn potentially facilitating treatment of conditions that would otherwise not be treatable using currently known methods or, at least, facilitating treatment of those conditions with methods that might be easier than those capable of carrying the uncompressed payloads. This may also allow treatments that would have previously required multiple vectors to deliver to instead be delivered in only one vector, reducing costs and easing treatment. These efforts are also not restricted to applications in medicine, but also provide similar benefits for delivery to plants, fungi, or animals for agricultural purposes and for delivery to microorganisms for biotechnological applications. Further, many delivery vectors and plasmids are easier to synthesize, clone, manufacture and/or otherwise work with when they are smaller, even if their maximum size is not exceeded. All of the above applications in medicine, agriculture, and biotechnology may also be eased through reductions in the sizes of the necessary components even in the absence of direct payload limits.

The following list of aspects reflects a variety of the embodiments explicitly contemplated by the present disclosure. Those of ordinary skill in the art will readily appreciate that the aspects below are neither limiting of the embodiments disclosed herein, nor exhaustive of all of the embodiments conceivable from the disclosure above, but are instead meant to be exemplary in nature.
1. A method of compressing genetic information in multiple reading frames by intersecting graph representations, the method comprising: for a first genetic sequence encoding a first protein or nucleic acid sequence, associating a first score with each possible nucleotide residue, insertion, and deletion at each position; encoding the first genetic sequence in a first computer-readable data structure comprising a first directed acyclic graph (DAG) or a first finite automaton (FA) such that (i) a plurality of potential genetic sequences for the first protein or nucleic acid sequence are encoded in the first data structure, (ii) each edge in the first DAG or first FA represents a nucleotide residue, insertion, or deletion at that position and the first score associated with the nucleotide residue, insertion, or deletion at that position, (iii) each path through the first DAG or accepted sequence in the first FA represents a potential sequence encoding the first protein or nucleic acid sequence, and (iv) for each path through the first DAG or accepted sequence in the first FA, a first aggregate score of the path or accepted sequence is the accumulation of the first score of all edges along the path or accepted sequence; encoding the additional genetic elements, such as proteins or nucleic acid sequences to be overlapped with the first sequence and with each other in additional first DAGs or first FAs; encoding, in a second DAG or a second FA, overlapping sequences between the encoded first genetic sequences for the first protein or nucleic acid sequences; calculating, for each edge in the second DAG or the second FA, a second score representing a combined total effect of the component edges of the first data structures; selecting, according to the score of each edge in the second DAG or second FA, a sequence represented by a path through the second DAG or the second FA.
2. The method according to aspect 1, wherein each score reflects a likelihood of the inclusion of a particular nucleotide residue, a particular insertion, or a particular deletion at the corresponding position.
3. The method according to aspect 1, wherein each score reflects a fitness metric associated with the inclusion of a particular nucleotide residue, a particular insertion, or a particular deletion at the corresponding position.
4. The method according to aspect 1, wherein each score reflects an expression of the probability of the inclusion of a particular nucleotide residue, a particular insertion, or a particular deletion at the corresponding position.
5. The method according to claim 1, wherein each score reflects an expression of the effect of the inclusion of a particular nucleotide residue, a particular insertion, or a particular deletion at the corresponding position.
6. The method according to any one of aspects 1 to 5, wherein encoding, in the second DAG or the second FA, overlapping sequences between the encoded genetic sequence for the first proteins or nucleic acid sequences comprises: selecting, in each of the first data structures, a starting position, the starting positions each having an edge representing the same nucleotide residue or an insertion or deletion; starting at the starting positions, adding to the second DAG or the second FA an edge each time the transitions between successive nodes in each of the first data structures include at least one overlapping nucleotide residue, insertion, or deletion, the added edge corresponding to the at least one overlapping nucleotide residue, insertion, or deletion.
7. The method according to any one of aspects 1 to 6, wherein encoding, in the second DAG or the second FA, overlapping sequences between the encoded genetic sequence for the first proteins or nucleic acid sequences comprises: trimming the second DAG or second FA by removing paths that end at nodes that are not end nodes of the first proteins or nucleic acid sequences, but have no valid outgoing paths.
8. The method according to any one of aspects 1 to 8, further comprising: evaluating the second DAG or the second FA according to a longest path algorithm to determine a potentially useful overlap sequence.
9. The method according to any one of aspects 1 to 8, further comprising: evaluating the second DAG or the second FA according to a shortest path algorithm to determine a potentially useful overlap sequence.
10. The method according to any one of aspects 1 to 9, further comprising: evaluating the second DAG or the second FA according to a stochastic algorithm to determine a potentially useful overlap sequence.
11. The method according to any one of aspects 1 to 10, further comprising: evaluating the second DAG or the second FA according to a deterministic algorithm to determine a potentially useful overlap sequence.
12. The method according to any one of aspects 1 to 11, further comprising: adjusting one or more scores associated with corresponding one or more edges of the second DAG or the second FA to promote or demote paths with specific attributes.
13. The method according to any one of aspects 1 to 12, further comprising: adjusting an overall score of the second DAG or the second FA to account for one or more non-local effects such as to interactions between residues.
14. The method according to aspect 13, wherein the non-local effects are determined according to mutagenesis or bioinformatics studies.
15. The method according to any one of aspects 1 to 14, further comprising experimentally testing the selected sequence.
16. The method according to any one of aspects 1 to 15, wherein: encoding overlapping sequences between the encoded genetic sequences for the first proteins or nucleic acid sequences comprises encoding in the second DAG, and the second DAG is isomorphic to a nondeterministic FA.
17. The method according to any one of aspects 1 to 15, wherein: encoding overlapping sequences between the encoded genetic sequences for the first protein or nucleic acid sequences comprises encoding in the second DAG, and the second DAG is isomorphic to a deterministic FA.
18. The method according to any one of aspects 1 to 17, further comprising: encoding, in a plurality of second DAGs or second FAs, overlapping sequences between the encoded genetic sequences for the first proteins or nucleic acid sequences, wherein each of the plurality of second DAGs or second FAs corresponds to a respective combination of starting positions in the first DAGs or FAs.
19. The method according to aspect 18, further comprising: generating a reverse complement of some number of the first DAGs or FAs; and encoding in one of the plurality of second DAGs or second FAs, overlapping sequences between first DAGs or FAs where some number of the first DAGs or first FAs are reverse complemented.
20. The method according to any one of aspects 1 to 19, wherein: the first proteins or nucleic acid sequences are proteins.
21. The method according to any one of aspects 1 to 19, wherein: the first proteins or nucleic acid sequences are nucleic acid sequences.
22. The method according to any one of aspects 1 to 19, wherein: the first proteins or nucleic acid sequences contain some nucleic acid sequences, and the some proteins.
23. A system operable to perform the method of any one of aspects 1 to 22.
24. A system comprising: a computer processor; a memory, communicatively coupled to the computer processor, the memory storing instructions, executable by the computer processor, and causing the processor to: for a first genetic sequence encoding a first protein or nucleic acid sequence, associate a first score with each possible nucleotide residue, insertion, and deletion at each position; encode the first genetic sequence in a first computer-readable data structure comprising a first directed acyclic graph (DAG) or a first finite automaton (FA) such that (i) a plurality of potential genetic sequences for the first protein or nucleic acid sequence are encoded in the first data structure, (ii) each edge in the first DAG or first FA represents a nucleotide residue, insertion, or deletion at that position and the first score associated with the nucleotide residue, insertion, or deletion at that position, (iii) each path through the first DAG or accepted sequence in the first FA represents a potential sequence encoding the first protein or nucleic acid sequence, and (iv) for each path through the first DAG or accepted sequence in the first FA, a first aggregate score of the path or sequence is the accumulation of the first score of all edges along the path or sequence; encoding the additional genetic elements, such as proteins or nucleic acid sequences to be overlapped with the first sequence and with each other in additional first DAGs or first FAs; encode, in a second DAG or a second FA, overlapping sequences between the encoded first genetic sequences for the first proteins or nucleic acid sequences; calculate, for each edge in the second DAG or the second FA, a second score representing a combined total effect of the component edges of the first data structures; select, according to a second aggregate score of each of the edges in the second data structure, a sequence represented by a path through the second DAG or the second FA.
25. A system comprising: a computer processor; a memory, communicatively coupled to the computer processor, the memory storing (i) data and (ii) instructions executable by the computer processor, the data and instructions comprising: a first routine operable to cause the computer processor to generate directed acyclic graphs (DAGs) or finite automatons (FAs) from protein mutation data; a second routine operable to cause the computer processor to create overlap DAGs or FAs using as input a series of DAGs or FAs output by the first routine; a third routine operable to cause the computer to evaluate an overlap DAG or FA to (i) locate best paths through the DAG or FA, and/or (ii) to alter edge weights according to specific criteria or stochastically before generating new best paths through the DAG or FA; and a fourth routine operable to cause the computer to score and optimize the best paths according to higher-order interactions.
26. The method according to any one of aspects 1 to 22, applied to enable delivery of a larger genetic payload than otherwise accepted by the vector.
27. A method of treatment employing the method of any one of aspects 1 to 22 to increase, for a particular vector, the payload size that can be delivered in the method of treatment.
28. A method of treatment employing the method of any one of aspects 1 to 22 to reduce the number of vectors required to deliver the cargo.
29. A method of treatment employing the method of any one of aspects 1 to 22 to ease the synthesis, manufacture, cloning, or use of a vector by reducing its total size

1. A method of compressing genetic information in multiple reading frames by intersecting graph representations, the method comprising:
for a series of first genetic sequences encoding first proteins or nucleic acid sequences, associating a first score with each possible nucleotide or amino acid residue, insertion, and deletion at each position;
encoding the first genetic sequences in first computer-readable data structures comprising first directed acyclic graphs (DAGs) or a first finite automatons (FAs) such that (i) a plurality of potential genetic sequences for the first proteins or nucleic acid sequences are encoded in the first data structures, (ii) each edge in the first DAGs or first FAs represents a nucleotide residue, insertion, or deletion at that position and the first score associated with the nucleotide residue, insertion, or deletion at that position, (iii) each path through the first DAGs or accepted sequence in the first FAs represents a potential sequence encoding one of the first proteins or nucleic acid sequences, and (iv) for each path through one of the first DAGs or accepted sequence in one of the first FAs, a first aggregate score of the path or accepted sequence is the accumulation of the first score of all edges along the path or accepted sequence;
encoding, in a second DAG or a second FA, overlapping sequences between the encoded first genetic sequences for the first proteins or nucleic acid sequences;
calculating, for each edge in the second DAG or the second FA, a second score representing a combined total effect of the component edges of the first data structures;
selecting, according to the scores of each edge in the second DAG or second FA, a sequence represented by a path through the second DAG or the second FA.

2. The method according to claim 1, wherein each score reflects a likelihood of the inclusion of a particular nucleotide residue, a particular insertion, or a particular deletion at the corresponding position.

3. The method according to claim 1, wherein each score reflects a fitness metric associated with the inclusion of a particular nucleotide residue, a particular insertion, or a particular deletion at the corresponding position.

4. The method according to claim 1, wherein each score reflects an expression of the probability of the inclusion of a particular nucleotide residue, a particular insertion, or a particular deletion at the corresponding position.

5. The method according to claim 1, wherein each score reflects an expression of the effect of the inclusion of a particular nucleotide residue, a particular insertion, or a particular deletion at the corresponding position.

6. The method according to claim 1, wherein encoding, in the second DAG or the second FA, overlapping sequences between the encoded genetic sequence for the first proteins or nucleic acid sequences comprises:
selecting, in each of the first data structures, a starting position for each data structure, the starting positions each having an edge representing the same nucleotide residue or an insertion or deletion;
starting at the starting positions, adding to the second DAG or the second FA an edge each time the transitions between successive nodes in each of the first data structures includes at least one overlapping nucleotide residue or an insertion or deletion, the added edge corresponding to the at least one overlapping nucleotide residue or an insertion or deletion.

7. The method according to claim 1, wherein encoding, in the second DAG or the second FA, overlapping sequences between the encoded genetic sequence for the first proteins or nucleic acid sequences comprises:
trimming the second DAG or second FA by removing paths that end at nodes that are not end nodes of any of the first proteins or nucleic acid sequences, but have no valid outgoing paths.

8. The method according to claim 1, further comprising:
evaluating the second DAG or the second FA according to a longest path algorithm to determine a potentially useful overlap sequence.

9. The method according to claim 1, further comprising:
evaluating the second DAG or the second FA according to a shortest path algorithm to determine a potentially useful overlap sequence.

10. The method according to claim 1, further comprising:
evaluating the second DAG or the second FA according to a stochastic algorithm to determine a potentially useful overlap sequence.

11. The method according to claim 1, further comprising:
evaluating the second DAG or the second FA according to a deterministic algorithm to determine a potentially useful overlap sequence.

12. The method according to claim 1, further comprising:
adjusting one or more scores associated with corresponding one or more edges of the second DAG or the second FA to promote or demote paths with specific attributes.

13. The method according to claim 1, further comprising:
adjusting an overall score of the second DAG or the second FA to account for one or more non-local effects attributed to interactions between residues.

14. The method according to claim 13, wherein the non-local effects are determined according to mutagenesis or bioinformatics studies.

15. The method according to claim 1, further comprising experimentally testing the selected sequence.

16. The method according to claim 1, wherein:
encoding overlapping sequences between the encoded genetic sequence for the first proteins or nucleic acid sequences comprises encoding in the second DAG, and
the second DAG is isomorphic to a nondeterministic FA.

17. The method according to claim 1, wherein:
encoding overlapping sequences between the encoded genetic sequence for the first proteins or nucleic acid sequences comprises encoding in the second DAG, and
the second DAG is isomorphic to a deterministic FA.

18. The method according to claim 1 further comprising:
encoding, in a plurality of second DAGs or second FAs, overlapping sequences between the encoded genetic sequence for the first proteins or nucleic acid sequences,
wherein each of the plurality of second DAGs or second FAs corresponds to a respective combination of starting positions in the first DAGs or FAs.

19. The method according to claim 18, further comprising:
generating reverse complements of some of the first DAGs or FAs; and
encoding in one of the plurality of second DAGs or second FAs, overlapping sequences between some combination of reverse complemented and non-reverse complemented first DAGs or FAs.

20. The method according to claim 1, wherein the first proteins or nucleic acid sequences are first proteins.

21. The method according to claim 1, wherein the first proteins or nucleic acid sequences are first nucleic acid sequences.

22. The method according to claim 1, wherein the first proteins or nucleic acid sequences are a combination of nucleic acid sequences and proteins.

23. A system comprising:
a computer processor;
a memory, communicatively coupled to the computer processor, the memory storing instructions, executable by the computer processor, and causing the processor to:
for a series of first genetic sequences encoding first proteins or nucleic acid sequences, associate a first score with each possible nucleotide or amino acid residue, insertion, and deletion at each position;
encode the first genetic sequences in first computer-readable data structures comprising first directed acyclic graphs (DAGs) or first finite automatons (FAs) such that (i) a plurality of potential genetic sequences for the first proteins or nucleic acid sequences are encoded in the first data structures, (ii) each edge in the first DAGs or first FAs represents a nucleotide residue, insertion, or deletion at that position and the first score associated with the nucleotide residue, insertion, or deletion at that position, (iii) each path through the first DAGs or accepted sequence in the first FAs represents a potential sequence encoding one of the first proteins or nucleic acid sequences, and (iv) for each path through one of the first DAGs or accepted sequence in one of the first FAs, a first aggregate score of the path or accepted sequence is the accumulation of the first score of all edges along the path or accepted sequence;
encode, in a second DAG or a second FA, overlapping sequences between the encoded first genetic sequences for the first proteins or nucleic acid sequences;
calculate, for each edge in the second DAG or the second FA, a second score representing a combined total effect of the component edges of the first data structures;
select, according to a second aggregate score of each of the edges, a sequence represented by a path through the second DAG or the second FA.

24. A system comprising:
a computer processor;
a memory, communicatively coupled to the computer processor, the memory storing (i) data and (ii) instructions executable by the computer processor, the data and instructions comprising:
a first routine operable to cause the computer processor to generate directed acyclic graphs (DAGs) or finite automatons (FAs) from protein mutation data;
a second routine operable to cause the computer processor to create overlap DAGs or FAs using as input a series of DAGs or FAs output by the first routine;
a third routine operable to cause the computer to evaluate an overlap DAG or FA to (i) locate best paths through the DAG or FA, and/or (ii) to alter edge weights according to specific criteria or stochastically before generating new best paths through the DAG or FA; and
a fourth routine operable to cause the computer to score and optimize the best paths according to non-local effects or higher-order interactions.
